(19) United States
(12) Patent Application Publication
(10) Pub. No.: US 2010/0240774 A1
(43) Pub. Date: Sep. 23, 2010

(54) USE OF HYDROPHOBIN POLYPEPTIDES AS PENETRATION ENHANCERS

(75) Inventors: Thomas Subkowski, Ladenburg (DE); Marvin Karos, Schwetzingen (DE); Heiko Barg, Weinheim (DE); Claus Bollschweiler, Heidelberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/721,973

(22) Filed: Mar. 11, 2010

Related U.S. Application Data
(63) Continuation-in-part of application No. PCT/EP2008/060870, filed on Aug. 20, 2008.

(30) Foreign Application Priority Data
Sep. 13, 2007 (EP) 07116363.8
Nov. 27, 2007 (EP) 07121650.1

(51) Int. Cl. A61K 47/42; A61K 8/64; A61P 17/00; A61P 43/00; A61Q 5/00; A61Q 11/00; A61Q 19/00; C07K 14/00; A01N 25/00

(52) U.S. Cl. 514/773; 530/350

(57) ABSTRACT

The present invention relates to the use of hydrophobin polypeptides as penetration intensifiers.

### RELATED APPLICATIONS

This application is a continuation-in-part of International Application PCT/EP2008/060870, filed Aug. 20, 2008, which claims benefit of European application 07116363.8, filed Sep. 13, 2007 and European application 07121650.1, filed Nov. 27, 2007.

### SUBMISSION OF SEQUENCE LISTING

The Sequence Listing associated with this application is filed in electronic format via EFS-Web and hereby is incorporated by reference into the specification in its entirety. The name of the text file containing the Sequence Listing is Replacement_Sequence_Listing_{—}13311_{—}00064_US.txt. The size of the text file is 73 KB; the text file was created on Jun. 1, 2010.

### BACKGROUND OF THE INVENTION

In recent years penetration intensifiers have achieved ever greater importance in a variety of fields, such as, for example, as a constituent of cosmetic or pharmaceutical compositions, of crop protection compositions or of coating compositions.

Transdermal penetration intensifiers are known from WO 93/002669. This describes a combination of polar and nonpolar penetration intensifiers in an active-ingredient-containing adhesive matrix in transdermal therapeutic systems. The polar penetration intensifiers specified are polyhydric alcohols, and the nonpolar penetration intensifiers specified are fatty acid esters. In the described transdermal therapeutic systems, the penetration intensifiers bring about an increase in the penetration rate of the active ingredient, which is steroid hormones that are insoluble or sparingly soluble in water.

In the transdermal therapeutic systems, the barrier function of the Stratum corneum is temporarily impaired by occlusion effects or by penetration intensifiers such as those mentioned above or dimethyl sulfoxide (DMSO), thus permitting the penetration of low molecular weight substances through the skin.

Further penetration intensifiers which are used in therapeutic preparations are, for example, mono- or polyhydric alcohols, such as ethanol, 1,2 propanediol or benzyl alcohol, saturated and unsaturated fatty alcohols having 8 to 10 carbon atoms, such as lauryl alcohol or cetyl alcohol, hydrocarbons, such a mineral oil, alkanes, esters, azones, such as 1-dodecylazacycloheptan-2-one, propylene glycol, chitosan, saturated and unsaturated fatty acids, such as stearic acid or oleic acid, fatty acid esters having up to 24 carbon atoms or dicarboxylic acid diesters having up to 24 carbon atoms, such as the methyl esters, ethyl esters, isopropyl esters, butyl esters, sec-butyl esters, isobutyl esters, tert-butyl esters and monoglyceric acid esters of acetic acid, caproic acid, lauric acid, myristic acid, stearic acid and palmitic acid, phosphate derivatives, such as lecithin, terpenes, urea and its derivatives and ethers, such as dimethyl isosorbide and diethylene glycol monoethyl ether, bile salts, polyethoxyethylenes, EDTA, nerolidol, limonene oxides or phospholipids.

Further known penetration intensifiers are SDS (sodium dodecylsulfate), dimethylformamide and N-methylformamide.

In the crop protection sector too, penetration intensifiers are used in order to ensure easier absorption of crop protection compositions.

Hydrophobins are small proteins of about 100 to 150 amino acids which occur in filamentous fungi, for example Schizophyllum commune. They usually have 8 cysteine units. Hydrophobins can be isolated from natural sources, but can also be obtained by means of genetic engineering methods, as disclosed, for example, by WO 2006/082251 or WO 2006/131564.

Hydrophobins are spread in a water-insoluble form on the surface of various fungal structures, such as e.g. aerial hyphae, spores, fruiting bodies. The genes for hydrophobins could be isolated from ascomycetes, deuteromycetes and basidiomycetes. Some fungi comprise more than one hydrophobin gene, e.g. Schizophyllum commune, Coprinus cinereus, Aspergillus nidulans. Different hydrophobins are evidently involved in different stages of fungal development. The hydrophobins here are presumably responsible for different functions (van Wetter et al., 2000, Mol. Microbiol., 36, 201-210; Kershaw et al. 1998, Fungal Genet. Biol, 1998, 23, 18-33).

As biological function for hydrophobins, besides the reduction in the surface tension of water for the generation of aerial hyphae, the hydrophobicization of spores is also described (Wösten et al. 1999, Curr. Biol., 19, 1985-88; Bell et al. 1992, Genes Dev., 6, 2382-2394). Furthermore, hydrophobins serve to line gas channels in fruiting bodies of lichen and as components in the recognition system of plant surfaces by fungal pathogens (Lugones et al. 1999, Mycol. Res., 103, 635-640; Hamer & Talbot 1998, Curr. Opinion Microbiol., volume 1, 693-697).

### DESCRIPTION OF RELATED ART

The use of hydrophobins for various applications has been proposed in the prior art.

WO 96/41882 proposes the use of hydrophobins as emulsifiers, thickeners, surface-active substances, for the hydrophilization of hydrophobic surfaces, for improving the water resistance of hydrophilic substrates, for producing oil-in-water emulsions and water-in-oil emulsions. Furthermore, pharmaceutical applications, such as the production of ointments or creams, and also cosmetic applications, such as skin protection or the protection of hair shampoos or hair rinses are proposed.

EP 1 252 516 discloses the coating of a variety of substrates, such as, for example, window, lens, biosensor, medical instrument, container, frame or automobile body, with a solution comprising hydrophobin at a temperature of from 30 to 80° C.

Furthermore, the use as demulsifier (WO 2006/103251), as evaporation retarder (WO 2006/128877) or soiling inhibitor (WO 2006/103215), for example, has been proposed.

US 20030217419A1 describes the use of the hydrophobin SC3 from Schizophyllumg commune for cosmetic preparations for the treatment of therapy materials. Here, cosmetic depots are formed which withstand several washes with shampoo.

The use of hydrophobins as penetration intensifier is hitherto not yet known.

### BRIEF SUMMARY OF THE INVENTION

The present invention relates to the use of hydrophobin polypeptides as penetration intensifiers.

### BRIEF DESCRIPTION OF THE SEVERAL VIEWS OF THE DRAWINGS

FIG. 1 illustrates that in two of three runs, the hydrophobins on their own bring about slightly reduced oxidative stress, the proteins on their own exhibit no reduction in oxidative stress in this run, whereas the combinations of the proteins and tocopherol acetate exhibit a drop.

FIG. 2 illustrates the results showing the effect of quercetin in combination with hydrophobin protein B was improved (H protein B 0.05%, combined with quercetin 0.0006%).

### DETAILED DESCRIPTION OF THE INVENTION

It was an object of the invention to provide a novel use for hydrophobin.

Additionally, it was the aim to achieve the object of allowing active ingredients to penetrate phase boundaries, or improving this, for which hitherto the phase boundary was impermeable or barely permeable.

It was a further object to lower the concentration of the active ingredients during the applications.

It was also an object of the present invention to provide cosmetic and/or pharmaceutical compositions comprising hydrophobin as penetration intensifier which ensure improved absorption of the active ingredients, in particular without causing irritation of the skin or mucosa.

It was a further object of the present invention to provide crop protection compositions comprising hydrophobin as penetration intensifier which ensure improved absorption of the active ingredients, in particular without causing damage to the plants treated or environmental damage.

The object is achieved through the use of hydrophobin as penetration intensifier.

Within the context of the invention, the terms “penetration intensifier” and “penetration promoter” are synonymous.

As regards the invention, the following can be stated specifically:

Penetration within the context of the present invention is the penetration of substances through a phase boundary.

Within the context of the present invention, a phase boundary is the transition from one phase to the adjacent phase.

Within the context of the present invention, a phase is an area within which no sharp change in any of its physical parameters occurs. Thus, during the transition from one phase to the adjacent phase, thus within a layer of only a few molecules in diameter, at least one physical or chemical property changes, selected from the group consisting of density, electric properties, magnetic properties, refractive index, chemical composition, crystal structure.

In one variant of the present invention, intensification of the penetration through a phase boundary means that, compared to a control which has the identical chemical, biological and physical properties, and under identical chemical, biological and physical conditions or prerequisites, a larger amount of active ingredients penetrates the phase boundary within the same time, or the same amount of active ingredients penetrates the phase boundary within a shorter time.

In one variant of the present invention, intensification of the penetration or increased penetration, or increased penetration of active ingredients through a phase boundary means that penetration of phase boundaries is facilitated or improved for active ingredients for which hitherto the phase boundary was impermeable or barely permeable. This compared to a control which has the identical chemical, biological and physical properties, and under identical chemical, biological and physical conditions or prerequisites, where a larger amount of active ingredients penetrates the phase boundary within the same time, or the same amount of active ingredients penetrates the phase boundary within a shorter time.

Penetration intensifiers are substances through which the penetration of another substance through a phase boundary is intensified.

Within the context of the present invention, the term “hydrophobin” or “hydrophobins” are to be understood hereinbelow as meaning polypeptides of the general structural formula (I)

Xₙ—C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I)

where X can be any of the 20 naturally occurring amino acids (Phe, Leu, Ser, Tyr, Cys, Trp, Pro, His, Gln, Arg, Ile Met, Thr, Asn, Lys, Val, Ala, Asp, Glu, Gly). Here, the radicals X may in each case be identical or different. Here, the indices alongside X are in each case the number of amino acids in the respective part sequence X, C is cysteine, alanine, serine, glycine, methionine or threonine, where at least four of the radicals named as C are cysteine, and the indices n and m, independently of one another, are natural numbers between 0 and 500, preferably between 15 and 300.

The polypeptides according to the formula (I) are also characterized by the property that, at room temperature, after coating a glass surface, they bring about an increase in the contact angle of a water drop of at least 8°, 10°, 20°, preferably at least 25° and particularly preferably 30°, in each case compared to the contact angle of a water drop of identical size with the uncoated glass surface.

The amino acids named as C¹ to C⁸ are preferably cysteines; however, they may also be replaced by other amino acids of similar space filling, preferably by alanine, serine, threonine, methionine or glycine. However, at least four, preferably at least 5, particularly preferably at least 6 and in particular at least 7, of the positions C¹ to C⁸ should consist of cysteines. In the proteins according to the invention, cysteines may either be present in reduced form or form disulfide bridges with one another. Particular preference is given to the intramolecular formation of C—C bridges, in particular those with at least one, preferably 2, particularly preferably 3 and very particularly preferably 4, intramolecular disulfide bridges. In the event of the above-described replacement of cysteines by amino acids of similar space filling, such C positions are advantageously exchanged in pairs which can form intramolecular disulfide bridges with one another.

If cysteines, serines, alanines, glycines, methionines or threonines are also used in the positions referred to with X, the numbering of the individual C positions in the general formulae can change accordingly.

Preference is given to using hydrophobins of the general formula (II)

Xₙ—C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

for carrying out the present invention, where X, C and the indices alongside X and C have the meaning above, the indices n and m are numbers between 0 and 350, preferably 15 to 300, the proteins are furthermore characterized by the above-mentioned change in contact angle, and furthermore at least 6 of the radicals named as C are cysteine. Particularly preferably, all of the radicals C are cysteine.

Particular preference is given to using hydrophobins of the general formula (III)

Xₙ—C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

where X, C and the indices alongside X have the meaning above, the indices n and m are numbers between 0 and 200, the proteins are furthermore characterized by the above-mentioned change in contact angle, and at least 6 of the radicals named as C are cysteine. Particularly preferably, all of the radicals C are cysteine.

The radicals Xₙ and Xₘ may be peptide sequences which are naturally also linked to a hydrophobin. However, it is also possible for one or both radicals to be peptide sequences which are naturally not linked to a hydrophobin. These are also to be understood as meaning those radicals Xₙ and/or Xₘ in which a peptide sequence naturally occurring in a hydrophobin is extended by a peptide sequence not naturally occurring in a hydrophobin.

If Xₙ and/or Xₘ are peptide sequences naturally not linked to hydrophobins, such sequences are generally at least 20, preferably at least 35, amino acids in length. These may be, for example, sequences of 20 to 500, preferably 30 to 400 and particularly preferably 35 to 100, amino acids.

Such a radical naturally not linked to a hydrophobin will also be referred to hereinbelow as fusion partner. This is intended to express that the proteins can consist of at least one hydrophobin part and one fusion partner part which do not occur together in this form in nature. Fusion hydrophobins composed of fusion partner and hydrophobin part have been disclosed, for example, in WO 2006/082251 (page 2, line 18 to page 5, line 25), WO 2006/082253 (page 2, line 20 to page 6, line 13) and WO 2006/131564 (page 2, line 17 to page 6, line 26).

The fusion partner part can be selected from a large number of proteins. It is possible for only a single fusion partner to be linked to the hydrophobin part, or for two or more fusion partners to be linked to a hydrophobin part, for example on the amino terminus (Xₙ) and on the carboxy terminus (Xₘ) of the hydrophobin part. However, it is also possible, for example, for two fusion partners to be linked to one position (Xₙ or Xₘ) of the protein according to the invention.

Particularly suitable fusion partners are proteins which occur naturally in microorganisms, in particular in E. coli or Bacillus subtilis. Examples of such fusion partners are the sequences yaad (SEQ ID NO: 16 in WO 2006/082251), yaae (SEQ ID NO: 18 in WO 2006/082251), ubiquitin and thioredoxin. Also highly suitable are fragments or derivatives of these specified sequences which comprise only part, for example 70 to 99%, preferably 5 to 50%, and particularly preferably 10 to 40%, of the specified sequences, or in which individual amino acids, or nucleotides have been altered compared to the specified sequence, the percentages referring in each case to the number of amino acids.

In a further preferred embodiment, the fusion hydrophobin also has, besides the specified fusion partner, as one of the groups Xₙ or Xₘ or as terminal constituent of such a group, a so-called affinity domain (affinity tag/affinity tail). In a manner known in principle, these are anchor groups which can interact with certain complementary groups and can serve for easier work-up and purification of the proteins. Examples of such affinity domains comprise (His)ₖ, (Arg)ₖ, (Asp)ₖ, (Phe)ₖ or (Cys)ₖ groups, where k is generally a natural number from 1 to 10. Preferably, it may be a (His)ₖ group, where k is 4 to 6. Here, the group Xₙ and/or Xₘ can consist exclusively of such a type of affinity domain or else a radical Xₙ or Xₘ, naturally linked or not naturally linked to a hydrophobin, is extended by a terminally arranged affinity domain.

The hydrophobins used according to the invention are hydrophobins according to the structural formulae (I), (II) and (III) and also fusion hydrophobins.

The hydrophobins used according to the invention can also be modified in their polypeptide sequence, for example by glycosylation, acetylation or else by chemical crosslinking, for example with glutardialdehyde.

One property of the hydrophobins used according to the invention or of their derivatives is the change in surface properties when the surfaces are coated with the proteins. The change in the surface properties can be determined experimentally, for example, by measuring the contact angle of a water drop before and after coating the surface with the protein and determining the difference between the two measurements.

The carrying out of contact angle measurements is known in principle to the person skilled in the art. The measurements refer to room temperature and also water drops of 5 μl and the use of small glass plates as substrate. The precise experimental conditions for a method, suitable by way of example, of measuring the contact angle are given in the experimental section. Under the conditions specified therein, the fusion proteins used according to the invention have the property of increasing the contact angle by at least 20°, preferably at least 25°, particularly preferably at least 30°; 40°, 45° in particular 50°, in each case compared with the contact angle of a water drop of identical size with the uncoated glass surface.

Particularly preferred hydrophobins for carrying out the present invention are the hydrophobins of the type dewA, rodA, hypA, hypB, sc3, basf1, basf2. These hydrophobins including their sequences are disclosed, for example, in WO 2006/82251. Unless stated otherwise, the sequences given below refer to sequences disclosed in WO 2006/82251 and herein. An overview table with the SEQ ID NOs: can be found in WO 2006/82251 on page 20 (line 1 to line 5).

Of particular suitability according to the invention are the fusion proteins yaad-Xa-dewA-his (SEQ ID NO: 20), yaad-Xa-rodA-his (SEQ ID NO: 22) or yaad-Xa-basf1-his (SEQ ID NO: 24) with the polypeptide sequences given in brackets, and also the nucleic acid sequences coding therefor, in particular the sequences according to SEQ ID NO: 19, 21, 23. Particularly preferably, yaad-Xa-dewA-his (SEQ ID NO: 20) can be used. Proteins which arise starting from the polypeptide sequences shown in SEQ ID NOs: 20, 22 or 24 as result of exchange, insertion or deletion of at least one, ranging to 10, preferably 5, particularly preferably 5%, of all amino acids, and which still have the biological property of the starting proteins to at least 50%, are also particularly preferred embodiments. Biological property of the proteins is to be understood here as meaning the already-described change in the contact angle by at least 20°, preferably at least 25°, particularly preferably at least 30°, 40°, 45°, in particular 50°.

Derivatives particularly suitable for carrying out the present invention are derivatives derived from yaad-Xa-dewA-his (SEQ ID NO: 20), yaad-Xa-rodA-his (SEQ ID NO: 22) or yaad-Xa-basf1-his (SEQ ID NO: 24) by shortening the yaad fusion partner. Instead of the complete yaad fusion partner (SEQ ID NO: 16) with 294 amino acids, a shortened yaad radical can advantageously be used. However, the shortened radical should comprise at least 20, preferably at least 35, amino acids. For example, a shortened radical with 20 to 293, preferably 25 to 250, particularly preferably 35 to 150 and for example 35 to 100, amino acids can be used. One example of such a protein is yaad40-Xa-dewA-his (SEQ ID NO: 26 in WO2007/014897; SEQ ID NO: 36 herein), which has a yaad radical shortened to 40 amino acids.

A cleavage site between the hydrophobin and the fusion partner or the fusion partners can be used to cleave off the fusion partner and to release the pure hydrophobin in underivatized form (for example by BrCN cleavage on methionine, factor Xa cleavage, enterokinase cleavage, thrombin cleavage, TEV cleavage etc.).

The hydrophobins used according to the invention as penetration intensifiers can be prepared chemically by known methods of peptide synthesis, such as, for example, by solid-phase synthesis in accordance with Merrifield.

Naturally occurring hydrophobins can be isolated from natural sources by means of suitable methods. By way of example, reference may be made to Wösten et. al., Eur. J. Cell Bio. 63, 122-129 (1994) or WO 96/41882 (page 23, line 15 to page 24, line 8).

A genetic engineering production method for hydrophobins without fusion partner from Talaromyces thermophilus is described by US 2006/0040349 (paragraphs [0071] to [0090]).

The preparation of fusion proteins can preferably take place by genetic engineering methods, in which a nucleic acid sequence coding for the fusion partner and a nucleic acid sequence coding for the hydrophobin part, in particular DNA sequence, are combined such that the desired protein is generated in a host organism through gene expression of the combined nucleic acid sequence. One such production method is disclosed, for example, by WO 2006/082251 (page 6, line 21 to page 12, line 37) or WO 2006/082253 (page 5, line 33 to page 11, line 13). The fusion partners make the production of the hydrophobins considerably easier. Fusion hydrophobins are produced in the genetic engineering methods with considerably better yields than hydrophobins without fusion partners.

The fusion hydrophobins produced from the host organisms by the genetic engineering method can be worked up in a manner known in principle and be purified by means of known chromatographic methods.

In one preferred embodiment, the simplified work-up and purification method disclosed in WO 2006/082253, pages 11/12 (page 11, line 15 to page 11, line 33) can be used.

For this, the fermented cells are firstly separated off from the fermentation broth, disrupted and the cell debris is separated off from the inclusion bodies. The latter can advantageously take place by centrifugation. Finally, the inclusion bodies can be disrupted in a manner known in principle by acids, bases and/or detergents in order to release the fusion hydrophobins. The inclusion bodies with the fusion hydrophobins used according to the invention can generally already be completely dissolved using 0.1 m NaOH within ca. 1 h.

The resulting solutions can—if appropriate after establishing the desired pH —be used for carrying out this invention without further purification. The fusion hydrophobins can, however, also be isolated as solid from the solutions. Preferably, the isolation can take place by means of spray granulation or spray drying, as described in WO 2006/082253, (page 11, line 35 to page 12, line 21). Besides remains of cell debris, the products obtained by the simplified work-up and purification method generally comprise ca. 80 to 90% by weight of proteins. The amount of fusion hydrophobins is generally 30 to 80% by weight, with regard to the amount of all of the proteins, depending on the fusion construct and fermentation conditions.

The isolated products comprising fusion hydrophobins can be stored as solids and be dissolved in the media desired in each case for use.

The fusion hydrophobins can be used as such or else after cleaving off and separating off the fusion partner as “pure” hydrophobins for carrying out this invention. A cleavage is advantageously carried out following isolation of the inclusion bodies and their dissolution.

According to the invention, the hydrophobins are used as penetration intensifiers.

In one embodiment, hydrophobin is used in combination with at least one further penetration intensifier, where at least one further penetration intensifier is selected from the group: DMSO, SDS (sodium dodecylsulfate), dimethylformamide, N-methylformamide, mono- or polyhydric alcohols, such as ethanol, 1,2-propanediol or benzyl alcohol, saturated and unsaturated fatty alcohols having 8 to 10 carbon atoms, such as lauryl alcohol or cetyl alcohol, hydrocarbons, such as mineral oil, alkanes, esters, azones, such as 1-dodecylazacycloheptan-2-one, propylene glycol, chitosan, saturated and unsaturated fatty acids, such as stearic acid or oleic acid, fatty acid esters having up to 24 carbon atoms or dicarboxylic acid diesters having up to 24 carbon atoms, such as the methyl esters, ethyl esters, isopropyl esters, butyl esters, sec-butyl esters, isobutyl esters, tert-butyl esters or monoglyceric acid esters of acetic acid, caproic acid, lauric acid, myristic acid, stearic acid and palmitic acid, phosphate derivates, such as lecithin, terpenes, urea and its derivatives and ethers, such as dimethyl isosorbide and diethylene glycol monoethyl ether, bile salts, polyethoxyethylenes, EDTA, nerolidol, limonene oxides or phospholipids.

In a further particularly preferred embodiment, hydrophobin is used as penetration intensifier in combination with DMSO or polyglycol.

In a further embodiment of the present invention, hydrophobin is used as penetration intensifier in combination with at least one further penetration intensifier in the care of leather and processing of leather.

In a further particularly preferred embodiment, hydrophobin is used as penetration intensifier for acids and bases, for example carboxylic acids or ammonia, buffer systems, polymers, inorganic particles such as SiO₂ or silicates, colorants such as, for example, dyes, fragrances or biocides in combination with at least one further penetration intensifier in the care of leather and processing of leather.

In a further embodiment of the present invention, the penetration through a phase boundary is intensified.

In a further embodiment, the penetration of active ingredients is promoted therethrough.

In one embodiment of the invention, active ingredients are to be understood as meaning all substances with a pharmaceutical or biological effect. Active ingredients are therefore compounds selected from the group consisting of pharmaceutically active compounds, therapeutically effective compounds and biologically active compounds, cosmetically active compounds, substance for supporting a cosmetic claim (for marketing purposes) such as pearl protein, which qualitatively and/or quantitatively influence, i.e. promote, or permit at all or inhibit, biochemical and/or physiological processes in an organism.

In addition, in small amounts, active ingredients develop a large pharmaceutical, chemical, biological or physiological effect.

Here, small amount is to be considered relative to the mass of the organism which the active ingredient reaches after penetrating the phase boundary.

This gives rise to a quotient of mass of the active ingredient penetrated through the phase boundary to the mass of the organism selected from the group consisting of the intervals: [1 ppt (1:10¹²) to 10% (1:10)], [1 ppb (1:10⁹ to 1% (1:100)], [1 ppt (1:10¹²) to 1 ppb (1:10^{9], [1} ppt (1:10¹² to 1:1000)], [1 ppb (1:10⁹ to 1:1000)], [1 ppt (1:10¹² to 1 ppm (1:10⁶)], [1 ppb (1:10⁹ to 1 ppm (1:10⁶)], [1 ppm (1:10⁶ to 1:1000)], [(1:1000) to 1% (1:100)].

In one variant of the present invention, an organism is selected from the group consisting of particular individuals from the kingdom of the protists, bacteria, fungi, plants or animals and also parts thereof such as cells and cell tissues.

In a further variant of the present invention, an organism is a dead organism or parts thereof, such as, for example, hide for producing leather.

By using hydrophobin as penetration intensifier, the intensification of the penetration of the active ingredient compared to the control can be 0.5; 0.6; 0.7; 0.9 or 1%. An intensification of the penetration by 2,3,4,5,6,7,8,9 or 10% is advantageous, an intensification of the penetration by 11,12,13,14 or 15% is particularly advantageous, and an intensification of the penetration by 16,17,18,19 or 20% or more % is very particularly advantageous.

The present invention further provides the use of hydrophobin for producing a composition for the improved absorption of active ingredients upon topical application.

A further field of use for the use according to the invention of hydrophobin as penetration intensifier is in the production of dermatological preparations.

In one embodiment, hydrophobin is thus used in a method for producing semisolid medicament forms or cosmetic preparations selected from the group consisting of ointment, cream, gel and paste.

The semisolid medicament forms are prepared as described for example in “Arzneiformelehre [Pharmacology]” by Ursula Schöffling, 4th edition, Deutsche Apotheker Verlag, 2003, pages 353 to 392.

Besides the auxiliaries described therein, the preparations comprise hydrophobin in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight, and also active ingredients in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight.

A further field of use for the use according to the invention of hydrophobin as penetration intensifier is in the production of agents for therapeutic or prophylactic use for certain diseases of the skin and mucosa. Fields of application therefore are in particular:
viral diseases (e.g. herpes, coxsackie, varicella zoster, cytomegalovirus etc.)
bacterial diseases (e.g. TB, syphilis etc.)
fungal diseases (e.g. candida, cryptococcus, histoplasmosis, aspergillus, mucormycosis etc.)
tumor diseases (e.g. melanomas, adenomas etc.)
autoimmune diseases (e.g. pemphigus vulgaris, bullous pemphi-goid, systemic lupus erythematosis etc.)
sunburn
parasitic attack (e.g. tics, mites, fleas etc.)
insect contact (e.g. blood-sucking insects such as anopheles etc.)

In one embodiment of the invention, the preparations for the aforementioned applications are in the form of aero dispersions, as described, for example, in “Arzneiformelehre [Pharmacology]” by Ursula Schöffling, 4th edition, Deutsche Apotheker Verlag, 2003, pages 336 to 352.

In one embodiment of the invention, the preparations for the applications specified above are in the form of release systems selected from the group consisting of nanoparticles, nanosuspensions, liposomes, microemulsion and bioadhesive preparation forms as described, for example, in “Arzneiformelehre [Pharmacology]” by Ursula Schöffling, 4th edition, Deutsche Apotheker Verlag, 2003, pages 468 to 471.

Besides the auxiliaries described therein, the preparations comprise hydrophobin in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight, and also active ingredients in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight.

A further field of application for the use according to the invention of hydrophobin as penetration intensifier is in the production of membranes, matrix or plasters comprising active ingredients, e.g. selected from the group consisting of transdermal therapeutic systems TTS.

These are prepared as described for example in “Arzneiformelehre [Pharmacology]” by Ursula Schöffling, 4th edition, Deutsche Apotheker Verlag, 2003, pages 462 to 468.

Besides the auxiliaries described therein, the preparations comprise hydrophobin in a fraction selected from the group consisting of 0.000001 to 30% by weight, 0.0001 to 30% by weight, 0.001 to 30% by weight, 0.01 to 30% by weight, 0.1 to 30% by weight and 1 to 30% by weight, and also active ingredients in a fraction selected from the group consisting of 0.000001 to 30% by weight, 0.0001 to 30% by weight, 0.001 to 30% by weight, 0.01 to 30% by weight, 0.1 to 30% by weight, 1 to 30% by weight and 0.1 to 50% by weight, 1 to 50% by weight.

A further embodiment for the use according to the invention of hydrophobin as penetration intensifier is in the production of cosmetic preparations.

In one variant, these are hair cosmetic, skin cosmetic or dental cosmetic preparations.

In the preparations or compositions specified according to the invention, in one embodiment of the present invention effector molecules can be used as active ingredients.

Effector molecules are understood hereinbelow as meaning molecules which have a certain predictable effect. These may either be protein-like molecules, such as enzymes, or non-proteinaceous molecules such as dyes, photoprotective agents, vitamins and fatty acids, or compounds comprising metal ions.

Among the protein-like effector molecules, preference is given to enzymes, peptides and antibodies.

Among the enzymes, the following are preferred as effector molecules: oxidases, peroxidases, proteases, tyrosinases, metal-binding enzymes, lactoperoxidase, lysozyme, amyloglycosidase, glucose oxidase, superoxide dismutase, photolyase, calalase.

Highly suitable protein-like effector molecules are also hydrolyzates of proteins from vegetable and animal sources, for example hydrolyzates of proteins of marine origin or silk hydrolyzates.

Of particularly good suitability are defined peptides which are used for antiaging, such as Matrixyl (INCI Name Glycerin-Water-Butylene Glycol-Carbomer-Polysorbate 20-Palmitoyl Pentapeptide-4), Argireline (INCI Name Aqua, Acety-Hexapeptide-3), Rigin (INCI Name Water (and)-Glycerin (and) Steareth-20 (and) Palmitoyltetrapeptide-7), Eyeliss (INCI Name Water-Glycerin-Hespiridin Methyl Chalcone-Steareth-20-Dipeptide-2-Palmitoyl Tetrapeptide-7), Regu-Age (INCI Name Oxido Reductases-Soy Peptides-Hydrilyzed Rice Bran Extract) and Melanostatin-5 (INCI Name Aqua-dextran-Nonapetide-1).

Among the non-protein-like effector molecules, preference is given to dyes, for example semipermanent dyes or oxidation dyes. Suitable dyes are all customary hair dyes for the molecules according to the invention. Suitable dyes are known to the person skilled in the art from cosmetics handbooks, for example Schrader, Grundlagen and Rezepturen der Kosmetika [Fundamentals and Formulations of Cosmetics], Hüthig Verlag, Heidelberg, 1989, ISBN 3-7785-1491-1.

Furthermore, antioxidants are preferred as effector molecules. Antioxidants, which are also referred to as free-radical scavengers, are able to neutralize so-called free radicals. These are aggressive compounds which are formed physiologically in numerous metabolic processes and the production of energy. They are important for defense reactions by the body, but can also bring about damage to genetic material (DNA), the cell membranes and body proteins. This damage can lead to premature tissue aging, tissue death and cancer. The antioxidants include carotenoids ascorbic acid (vitamin C, E 300) and also sodium L-ascorbate (E 301) and calcium L-ascorbate (E 302); ascorbyl palmitate (E 304); butylhydroxyanisol (E 320); butylhydroxytoluene (E 321); calcium-disodium-EDTA (E 385); gallate and also propyl gallate (E 310), octyl gallate (E 311) and dodecyl gallate (lauryl gallate) (E 312); isoascorbic acid (E 315) and also sodium isoascorbate (E 316); lecithin (E 322); lactic acid (E 270); multi-phosphates such as diphosphates (E 450), triphosphates (E 451) and polyphosphates (E 452); sulfur dioxide (E 220) and also sodium sulfite (E 221), sodium bisulfite (E 222), sodium disulfite (E 223), potassium sulfite (E 224), calcium sulfite (E 226), calcium hydrogensulfite (E 227) and potassium bisulfite (E 228); selenium; tocopherol (vitamin E, E 306) and also alpha-tocopherol (E 307), gamma-tocopherol (E 308) and delta-tocopherol (E 309); tin II tin II tin II tin II chloride (E 512); citric acid (E 330) and also sodium citrate (E 331) and potassium citrate (E 332); L-gluthathione, L-cysteine, polyphenols, flavonoids, phytoestrogens, glutathione and the antioxidative enzymes superoxide dismutase, glutathione peroxidase and catalase.

According to the invention, as antioxidants, at least one compound is selected from the aforementioned group of antioxidants.

Further suitable effector molecules are carotenoids. According to the invention, carotenoids are to be understood as meaning the following compounds: beta-carotene, lycopene, lutein, astaxanthin, zeaxanthin, cryptoxanthin, citranaxanthin, canthaxanthin, bixin, beta-Apo-4-carotenal, beta-Apo-8-carotenal, beta-Apo-8-carotenoic acid ester, individually or as mixture.

Preferably used carotenoids are beta-carotene, lycopene, lutein, astaxanthin, zeaxanthin, citranaxanthin and canthaxanthin.

Within the context of the present invention, retinoids mean vitamin A alcohol (retinol) and its derivatives, such as vitamin A aldehyde (retinal), vitamin A acid (retinoic acid) and vitamin A ester (e.g. retinyl acetate, retinyl propionate and retinyl palmitate). The term retinoic acid here comprises both all-trans retinoic acid and also 13-cis retinoic acid. The terms retinol and retinal preferably comprise the all-trans compounds. A preferred retinoid used for the suspensions according to the invention is all-trans retinol, referred to below as retinol.

Further preferred effector molecules are vitamins, in particular vitamins A and esters thereof.

Vitamins are essential organic compounds which are either not synthesized or synthesized only in inadequate amounts in the animal and human organism. On the basis of this definition, 13 components or groups of components have been classified as vitamins. The fat-soluble vitamins include vitamin A (retinols), vitamin D (calciferols), vitamin E (tocopherols, tocotrienols) and vitamin K (phylloquinones). The water-soluble vitamins include vitamin B₁ (thiamine), vitamin B₂ (riboflavin), vitamin B₆ (pyridoxal group), vitamin B₁₂ (cobalamine), vitamin C (L-ascobic acid), pantothenic acid, biotin, folic acid and niacin.

Vitamins, provitamins and vitamin precursors from the groups A, C, E and F, in particular 3,4-didehydroretinol, beta-carotene (provitamin of vitamin A), ascorbic acid (vitamin C), and the palmitic acid esters, glucosides or phosphates of ascorbic acid, tocopherols, in particular atocopherol, and its esters, e.g. the acetate, the nicotinate, the phosphate and the succinate; also vitamin F, which is understood as meaning essential fatty acids, particularly linoleic acid, linolenic acid and arachidonic acid.

Vitamin E is a collective term for a group of (to date) eight fat-soluble substances with antioxidative and nonantioxidative effects. Vitamin E is a constituent of all membranes of animal cells, but is formed only by photosynthetically active organisms such as plants and cyanobacteria. Four of the eight known vitamin E forms are tocopherols (alpha-tocopherol, beta-tocopherol, gamma-tocopherol and delta-tocopherol). The other hitherto known four forms of vitamin E are called tocotrienols (alpha-tocotrienol, beta-tocotrienol, gamma-tocotrienol and delta-tocotrienol). In addition, derivatives of these substances, such as alpha-tocopheryl acetate, may also be advantageous.

Vitamin A and its derivatives and provitamins advantageously exhibit a particular skin-smoothing effect.

The vitamins, provitamins, or vitamin precursors of the vitamin B group or derivatives thereof and the derivatives of 2-furanone to be used preferably according to the invention include inter alia:
vitamin B₁, trivial name thiamine, chemical name 3-[(4′-amino-2′-methyl-5′-pyrimidinyl)methyl]-5-(2-hydroxyethyl)-4-methylthiazolium chloride.
vitamin B₂, trivial name riboflavin, chemical name 7,8-dimethyl-10-(1-D-ribityl)benzo[g]pteridine-2,4(3H,10H)-dione. In free form, riboflavin occurs e.g. in whey, other riboflavine derivatives can be isolated from bacteria and yeasts. A stereoisomer of riboflavine which is likewise suitable according to the invention is lyxoflavin, which can be isolated from fishmeal or liver and carries a D-arabityl radical instead of the D-ribityl.
Vitamin B₃. This name is often used for the compounds nicotinic acid and nicotinamide (niacinamide). According to the invention, preference is given to nicotinamide.
Vitamin B₅ (pantothenic acid and panthenol). Preference is given to using panthenol. Derivatives of panthenol which can be used according to the invention are, in particular, the esters and ethers of panthenol, and cationically derivatized panthenols. In a further preferred embodiment of the invention, derivatives of 2-furanone can also be used in addition to pantothenic acid or panthenol. Particularly preferred derivatives are the also commercially available substances dihydro-3 hydroxy-4,4-dimethyl-2(3H)-furanone with the trivial name pantolactone (Merck), 4 hydroxymethyl-γ-butyrolactone (Merck), 3,3-dimethyl-2-hydroxy-g-butyrolactone (Aldrich) and 2,5-dihydro-5-methoxy-2-furanone (Merck), with all of the stereoisomers being expressly included.
Vitamin B₆, which is understood as meaning not a uniform substance, but the derivatives of 5-hydroxymethyl-2-methylpyridin-3-ol known under the trivial names pyridoxine, pyridoxamine and pyridoxal.
Vitamin B₇ (biotin), also referred to as vitamin H or “skin vitamin”. Biotin is (3aS,4S,6aR)-2-oxohexahydrothienol[3,4-d]imidazole-4-valeric acid.

Panthenol, pantolactone, nicotinamide and biotin are very particularly preferred according to the invention.

According to the invention, suitable derivatives (salts, esters, sugars, nucleotides, nucleosides, peptides and lipids) can be used.

As lipophilic, oil-soluble antioxidants from this group, preference is given to tocopherol and derivatives thereof, gallic acid esters, flavonoids and carotenoids, and also butylhydroxytoluene/anisole. Preferred water-soluble antioxidants are amino acids, e.g. tyrosine and cysteine and derivatives thereof, and also tannins, in particular those of vegetable origin.

Triterpenes, in particular triterpenoic acids, such as ursolic acid, rosmaric acid, betulinic acid, boswellic acid and bryonolic acid.

Further preferred effector molecules are preferably low-dose fruit acids (alpha-hydroxy acids), such as, for example, malic acid, citric acid, lactic acid, tartaric acid, glycolic acid. According to the invention, at least one compound from the aforementioned group of fruit acids is selected as effector molecules.

These may be present in concentrations of from 0.1% to 35%, preferably 0.1% to 10%, in particular 1% to 10%, 1% to 5%.

Further preferred effector molecules are urea and derivatives thereof. These may be present in concentrations of from 0.1% to 25%, preferably 0.1% to 10%, in particular 1% to 10%, 1% to 5%.

In one embodiment of the present invention, the effector molecules are joined to the hydrophobin polypeptide sequence.

The effector molecules are joined to a hydrophobin polypeptide sequence. The bond between effector molecules and hydrophobin polypeptide sequence may either be covalent or else based on ionic or van der Waals interactions.

Preference is given to a covalent linkage. This can take place for example via the side chains of the hydrophobin polypeptide sequence, in particular via amino functions or carboxylate functions or thiol functions. Preference is given to a linkage via the amino functions of one or more lysine radicals, one or more thiol group of cysteine radicals or via the N-terminal or C-terminal function of the hydrophobin polypeptide. The linkage of the effector molecules with the hydrophobin polypeptide sequence can take place either directly, i.e. as covalent linkage of two chemical functions already present in the effector molecule and the hydrophobin polypeptide sequence, for example an amino function of the hydrophobin polypeptide sequence is linked with a carboxylate function of the effector molecule to the acid amide. The linkage can, however, also be via a so-called linker, i.e. an at least bifunctional molecule which enters into a bond with one function of the hydrophobin polypeptide sequence and is linked with another function of the effector molecule.

If the effector molecule likewise consists of a polypeptide sequence, the linkage of effector molecules and hydrophobin polypeptide sequence can take place through a so-called fusion protein, i.e. a continuous polypeptide sequence which consists of the two part sequences, i.e. of effector molecules and hydrophobin polypeptide sequence.

It is also possible for so-called spacer elements to be incorporated between effector molecules and hydrophobin polypeptide sequence, for example polypeptide sequences which have a potential cleavage site for a protease, lipase, esterase, phosphatase, hydrolase, or polypeptide sequences which permit simple purification of the fusion protein, for example so-called His tags, i.e. oligohistidine radicals.

The linkage in the case of a nonprotein-like effector molecule with the hydrophobin polypeptide sequence preferably takes place through functionizable radicals (side groups) on the hydrophobin polypeptide, which enter into a covalent bond with a chemical function of the effector molecule.

Preference is given here to a bond linkage via an amino, thiol or hydroxy function of the hydrophobin polypeptide which can, for example with a carboxyl function of the effector molecule, optionally after activation, enter into a corresponding amide, thioester or ester bond.

A further preferred linkage of the hydrophobin polypeptide sequence with an effector molecule is the use of a tailored linker. Such a linker has two or more so-called anchor groups with which it can link the hydrophobin polypeptide sequence and one or more effector molecules. For example, an anchor group for hydrophobin peptide may be a thiol function, by means of which the linker can enter into a disulfide bond with a cysteine radical of the hydrophobin polypeptide. An anchor group for the effector molecule may be, for example, a carboxyl function, by means of which the linker can enter into an ester bond with a hydroxyl function of the effector molecule.

The use of such tailored linkers permits the precise matching of the linking to the desired effector molecule. Moreover, it is thereby possible to link a plurality of effector molecules with a hydrophobin polypeptide sequence in a defined manner.

The linker used is governed by the functionality to be coupled. Of suitability are, for example, molecules which couple to hydrophobin polypeptides by means of sulfhydryl-reactive groups, e.g. maleimides, pydridyldisulfides, alpha-haloacetyls, vinylsulfone and to effector molecules by means of
sulfhydryl-reactive groups, e.g. maleimides, pydridyldisulfides, alpha-haloacetyls, vinylsulfones), amine-reactive groups (e.g. succinimidyl esters, carbodiimides, hydroxymethylphosphine, imido esters, PFP esters etc.)
sugars and oxidized sugar-reactive groups (e.g. hydrazides etc.)
carboxy-reactive groups (e.g. carbodiimides etc.)
hydroxyl-reactive groups (e.g. isocyanates etc.)
thymine-reactive groups (e.g. psoralene etc.)
unselective groups (e.g. aryl azides etc.)
photoactivatable groups (e.g. perfluorophenyl azide etc.)
metal-complexing groups (e.g. EDTA, hexahis, ferritin)
antibodies and antibody fragments (e.g. single-chain antibodies, F(ab) fragments of antibodies, catalytic antibodies).

Alternatively, a direct coupling can be carried out between active ingredient/effect substance and the keratin binding domains, e.g. by means of carbodiimides, glutardialdehyde or other crosslinkers known to the person skilled in the art.

The linker may be stable, thermocleavable, photocleavable or else enzymatically cleavable (especially by lipases, esterases, proteases, phosphatases, hydrolases etc.). Corresponding chemical structures are known to the person skilled in the art and are integrated between the parts of the molecule.

Examples of enzymatically cleavable linkers which can be used in the molecules according to the invention are specified, for example, in WO 98/01406 (page 3, line 30 to page 23, line 9), to the entire contents of which reference is hereby expressly made.

The preparations according to the invention comprising hydrophobin as penetration intensifier have a relatively wide field of application in human cosmetics, in particular skincare and haircare, dental care, animal care, leather care and leather working.

Preferably, the preparations are used for skin, nail, dental and hair cosmetics. They permit a high concentration and long action time of skincare, nail care, dental and haircare or skin-protecting, nail-protecting, dental-protecting and hair-protecting effector substances.

Suitable auxiliaries and additives for producing hair cosmetic, dental cosmetic or skin cosmetic preparations are known to the person skilled in the art and can be found in cosmetics handbooks, for example Schrader, Grundlagen and Rezepturen der Kosmetika [Fundamentals and Formulations of Cosmetics], Hüthig Verlag, Heidelberg, 1989, ISBN 3-7785-1491-1.

According to a further embodiment, this hair cosmetic or skin cosmetic or dental cosmetic preparation serves for the care or the protection of the skin or hair or teeth and is in the form of an emulsion, a dispersion, a suspension, an aqueous surfactant preparation, a milk, a lotion, a cream, a balm, an ointment, a gel, granules, a powder, a stick preparation, such as e.g. a lipstick, a foam, an aerosol or a spray. Such formulations are highly suitable for topical preparations. Suitable emulsions are oil-in-water emulsions (O/W type) and water-in-oil emulsions (W/O type) or microemulsions.

As a rule, the hair cosmetic, dental cosmetic or skin cosmetic preparation is used for application to the skin (topical), teeth or hair. Topical preparations are to be understood here as meaning those preparations which are suitable for applying the active ingredients to the skin in fine distribution and preferably in a form which can be absorbed by the skin. Of suitability for this are, for example, aqueous and aqueous-alcoholic solutions, sprays, foams, foam aerosols, ointments, aqueous gels, emulsions of the O/W or W/O type, microemulsions or cosmetic stick preparations.

According to a preferred embodiment of the cosmetic composition according to the invention, the composition comprises a carrier. A preferred carrier is water, a gas, a water-based liquid, an oil, a gel, an emulsion or microemulsion, a dispersion or a mixture thereof. The specified carriers exhibit good skin compatibility. Aqueous gels, emulsions or microemulsions are particularly advantageous for topical preparations.

Emulsifiers which can be used are nonionogenic surfactants, zwitterionic surfactants, ampholytic surfactants or anionic emulsifiers. The emulsifiers may be present in the composition according to the invention in amounts of from 0.1 to 10% by weight, preferably 1 to 5% by weight, based on the composition.

A nonionogenic surfactant which may be used is, for example, a surfactant from at least one of the following groups:

Addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C_{12/18}-fatty acid mono- and diesters of addition products of from 1 to 30 mol of ethylene oxide onto glycerol;
glycerol mono- and diesters and sorbitan mono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethoxylated analogues thereof;
addition products of from 15 to 60 mol of ethylene oxide onto castor oil and/or hydrogenated castor oil;
polyol esters and in particular polyglycerol esters, such as, for example, polyglycerol polyricinoleate, polyglycerol poly-12-hydroxystearate or polyglycerol dimerate. Mixtures of compounds from two or more of these classes of substance are likewise suitable;
addition products of from 2 to 15 mol of ethylene oxide onto castor oil and/or hydrogenated castor oil;
partial esters based on linear, branched, unsaturated or saturated C_{6/22} fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose);
mono-, di- and trialkyl phosphates, and mono-, di- and/or tri-PEG alkyl phosphates and salts thereof;
wool wax alcohols;
polysiloxane-polyalkyl-polyether copolymers or corresponding derivatives;
mixed esters of pentaerythritol, fatty acids, citric acid and fatty alcohol as in DE-C 1165574 and/or mixed esters of fatty acids having 6 to 22 carbon atoms, methylglucose and polyols, preferably glycerol or polyglycerol, and
polyalkylene glycols;
betaines.

Furthermore, zwitterionic surfactants can be used as emulsifiers. Zwitterionic surfactants is the term used to refer to those surface-active compounds which carry at least one quaternary ammonium group and at least one carboxylate group or one sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and cocoacylaminoethylhydroxyethyl carboxymethylglycinate. Particular preference is given to the fatty acid amide derivative known under the CTFA name Cocamidopropyl Betaine.

Likewise suitable emulsifiers are ampholytic surfactants. Ampholytic surfactants are understood as meaning surface-active compounds which, apart from a C_{8,18}-alkyl or -acyl group in the molecule, contain at least one free amino group and at least one —COON or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group.

Particularly preferred ampholytic surfactants are N-cocoalkylaminopropionate, cocoacylaminoethylaminopropionate and C_{12/18}-acylsarcosine. Besides the ampholytic emulsifiers, quaternary emulsifiers are also suitable, with those of the esterquat type, preferably methyl-quaternized difatty acid triethanolamine ester salts, being particularly preferred. Furthermore, anionic emulsifiers which can be used are alkyl ether sulfates, monoglyceride sulfates, fatty acid sulfates, sulfosuccinates and/or ether carboxylic acids.

Suitable oil bodies are Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as e.g. propylene glycol, dimerdiol or trimertriol) and/or Guerbet alcohols, triglycerides based on C₆-C₁₀-fatty acids, liquid mono-/di-, triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcohol carbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. Oil bodies which can be used are also silicone compounds, for example dimethylpolysiloxanes, methylphenylpolysiloxanes, cyclic silicones, and amino-, fatty acid-, alcohol-, polyether-, epoxy-, fluorine-, alkyl- and/or glycoside-modified silicone compounds, which may be liquid or else resin-like at room temperature. The oil bodies may be present in the compositions according to the invention in amounts of from 1 to 90% by weight, preferably 5 to 80% by weight and in particular 10 to 50% by weight, based on the composition.

Suitable effector molecules (ii) for deodorants in particular are: perfume oils, cyclodextrins, ion exchangers, zinc ricinoleate, antimicrobial/bacteriostatic compounds (e.g. DCMX, Irgasan DP 300, TCC).

The following are suitable for antiperspirants: tannins, and zinc/aluminum salts.

Besides the described auxiliaries, the preparations comprise hydrophobin in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight.

In one embodiment of the present invention, hydrophobin is used as penetration intensifier in crop protection compositions.

The present invention further provides a process for the preparation of crop protection compositions comprising hydrophobin, and also crop protection compositions comprising hydrophobin.

Besides the described auxiliaries, the crop protection compositions comprise hydrophobin in a fraction selected from the group consisting of 0.000001 to 10% by weight, 0.0001 to 10% by weight, 0.001 to 10% by weight, 0.01 to 10% by weight, 0.1 to 10% by weight and 1 to 10% by weight.

The content of active ingredient and/or effect substance can be varied over wide ranges. In particular, the amphiphilic polymer compositions permit the preparation of so-called active ingredient concentrates which comprise the active ingredient in an amount of at least 5% by weight, e.g. in an amount of from 5 to 50% by weight and in particular in an amount of from 5 to 20% by weight, based on the total weight of the composition.

Advantageously, the aqueous active ingredient compositions according to the invention can be formulated to be solvent-free or low-solvent, i.e. the fraction of organic solvents in the aqueous active ingredient composition is often not more than 10% by weight, in particular not more than 5% by weight and in particular not more 1% by weight, based on the total weight of the composition.

A large number of different active ingredients and effect substances can be formulated in the aqueous compositions according to the invention. A particular embodiment of the invention relates to the formulation of active ingredients for crop protection, i.e. of herbicides, fungicides, nematicides, acaricides, insecticides, and also active ingredients which regulate plant growth.

Examples of fungicidal active ingredients which can be formulated as aqueous active ingredient composition according to the invention include:
acylalanines such as benalaxyl, metalaxyl, ofurace, oxadixyl;
amine derivates such as aldimorph, dodine, dodemorph, fenpropimorph, fenpropidin, guazatine, iminoctadine, spiroxamin, tridemorph;
anilinopyrimidines such as pyrimethanil, mepanipyrim or cyrodinyl;
antibiotics such as cycloheximide, griseofulvin, casugamycin, natamycin, polyoxin and streptomycin;
azoles such as bitertanol, bromoconazole, cyproconazole, difenoconazole, dinitroconazole, epoxiconazole, fenbuconazole, fluquiconazole, flusilazole, flutriafol, hexaconazole, imazalil, ipconazole, metconazole, myclobutanil, penconazole, propiconazole, prochloraz, prothioconazole, tebuconazole, tetraconazole, triadimefon, triadimenol, triflumizole, triticonazole;
2-methoxybenzophenones, as are described in EP-A 897904 by the general formula I, e.g. metrafenone;
dicarboximides such as iprodione, myclozolin, procymidone, vinclozolin;
dithiocarbamates such as ferbam, nabam, maneb, mancozeb, metam, metiram, propineb, polycarbamate, thiram, ziram, zineb;
heterocyclic compounds such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobezamid, probenazole, proquinazid, pyrifenox, pyroquilon, quinoxyfen, silthiofam; thiabendazole, thifluzamid, thiophanate-methyl, tiadinil, tricyclazole, triforine;
nitrophenyl derivatives such as binapacryl, dinocap, dinobuton, nitrophthal-isopropyl;
phenylpyrroles such as fenpiclonil and fludioxonil;
unclassified fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin acetate, fenoxanil, ferimzone, fluazinam, fosetyl, fosetyl aluminum, iprovalicarb, hexachlorobenzene, metrafenone, pencycuron, propamocarb, phthalide, toloclofos-methyl, quintozene, zoxamide;
strobilurins as described in WO 03/075663 by the general formula I, for example azoxystrobin, dimoxystrobin, fluoxastrobin, kresoxim-methyl, metominostrobin, orysastrobin, picoxystrobin, pyraclostrobin and trifloxystrobin;
sulfenic acid derivatives such as captafol, captan, dichlofluanid, folpet, tolylfluanid;
cinnamides and analogues such as dimethomorph, flumetover, flumorp;
6-aryl-[1,2,4]triazolo[1,5-a]pyrimidines as described e.g. in WO 98/46608, WO 99/41255 or WO 03/004465 (in each case by the general formula I page 1, line 8 to page 11, line 45, and also compounds depicted in formula IA in conjunction with tables 1 to 44 and table A in WO 03/00465);
amide fungicides such as cyclofenamid and also (Z)-N-[α-(cyclopropylmethoxyimino)-2,3-difluoro-6-(difluoromethoxy)benzyl]-2-phenylacetamide.

Examples of herbicides which can be formulated as aqueous active ingredient composition according to the invention include:
1,3,4-thiadiazoles such as buthidazole and cyprazole;
amides such as allidochlor, benzoylpropethyl, bromobutide, chlorthiamid, dimepiperate, dimethenamid, diphenamid, etobenzanid, flampropmethyl, fosamin, isoxaben, metazachlor, alachlor, acetochlor, metolachlor, monalide, naptalam, pronamid, propanil;
aminophosphoric acids such as bilanafos, buminafos, glufosinate ammonium, glyphosate, sulfosate;
aminotriazoles such as amitrol, anilides such as anilofos, mefenacet;
aryloxyalkanoic acids such as 2,4-D, 2,4-DB, clomeprop, dichlorprop, dichlorprop-P, fenoprop, fluoroxypyr, MCPA, MCPB, mecoprop, mecoprop-P, napropamide, napropanilide, triclopyr;
benzoic acids such as chloramben, dicamba;
benzothiadiazinones such as bentazone;
bleachers such as clomazone, diflufenican, fluorochloridone, flupoxam, fluridone, pyrazolate, sulcotrione;
carbamates such as carbetamid, chlorbufam, chlorpropham, desmedipham, phenmedipham, vernolate;
quinolinic acids such as quinclorac, quinmerac;
dichloropropionic acids such as dalapon;
dihydrobenzofurans such as ethofumesate;
dihydrofuran-3-one such as flurtamone;
dinitroanilines such as benefin, butralin, dinitramine, ethalfiuralin, fluchloralin, isopropalin, nitralin, oryzalin, pendimethalin, prodiamine, profluralin, trifluralin, dinitrophenols such as bromofenoxim, dinoseb, dinoseb acetate, dinoterb, DNOC, minoterb acetate;
diphenyl ethers such as acifluorfen-sodium, aclonifen, bifenox, chlornitrofen, difenoxuron, ethoxyfen, fluorodifen, fluoroglycofen-ethyl, fomesafen, furyloxyfen, lactofen, nitrofen, nitrofluorfen, oxyfluorfen;
dipyridyls such as cyperquat, difenzoquat methyl sulfate, diquat, paraquat dichloride;
imidazoles such as isocarbamid;
imidazolinones such as imazamethapyr, imazapyr, imazaquin, imazethabenz-methyl, imazethapyr, imazapic, imazamox;
oxadiazoles such as methazole, oxadiargyl, oxadiazon;
oxiranes such as tridiphane;
phenols such as bromoxynil, ioxynil;
phenoxyphenoxypropionic acid esters such as clodinafop, cyhalofop-butyl, diclofop-methyl, fenoxaprop-ethyl, fenoxaprop-p-ethyl, fenthiapropethyl, fluazifop-butyl, fluazifop-p-butyl, haloxyfop-ethoxyethyl, haloxyfop-methyl, haloxyfop-p-methyl, isoxapyrifop, propaquizafop, quizalofop-ethyl, quizalofop-p-ethyl, quizalofop-tefuryl;
phenylacetic acids such as chlorfenac;
phenylpropionic acids such as chlorophenprop-methyl;
ppi active ingredients such as benzofenap, cinidon-ethyl, flumiclorac-pentyl, flumioxazin, flumipropyn, flupropacil, pyrazoxyfen, sulfentrazone, thidiazimin;
pyrazoles such as nipyraclofen;
pyridazines such as chloridazon, maleic hydrazide, norflurazon, pyridate;
pyridinecarboxylic acids such as clopyralid, dithiopyr, picloram, thiazopyr;
pyrimidyl ethers such as pyrithiobac acid, pyrithiobac-sodium, KIH-2023, KIH-6127;
sulfonamides such as flumetsulam, metosulam,
triazolecarboxamides such as triazofenamid;
uracils such as bromacil, lenacil, terbacil;
also benazolin, benfuresate, bensulide, benzofluor, bentazon, butamifos, cafenstrole, chlorthal-dimethyl, cinmethylin, dichlobenil, endothall, fluorbentranil, mefluidide, perfluidone, piperophos, topramezone and prohexanedione-calcium;
sulfonylureas such as amidosulfuron, azimsulfuron, bensulfuron-methyl, chlorimuron-ethyl, chlorsulfuron, cinosulfuron, cyclosulfamuron, ethametsulfuron-methyl, flazasulfuron, halosulfuron-methyl, imazosulfuron, metsulfuron-methyl, nicosulfuron, primisulfuron, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron;
crop protection active ingredients of the cyclohexenone type such as alloxydim, clethodim, cloproxydim, cycloxydim, sethoxydim and tralkoxydim. Very particularly preferred herbicidal active ingredients of the cyclohexenone type are: tepraloxydim (cf. AGROW, No. 243, 3.11.95, page 21, caloxydim) and 2-(1-[2-{4-chlorophenoxy}propyl-oxyimino]butyl)-3-hydroxy-5-(2H-tetrahydrothiopyran-3-yl)-2-cyclohexen-1-one and of the sulfonylurea type: N-(((4-methoxy-6-[trifluoromethyl]-1,3,5-triazin-2-yl)amino)carbonyl)-2-(trifluoromethyl)benzenesulfonamide.

Examples of insecticides which can be formulated as aqueous active ingredient composition according to the invention comprise:
organophosphates such as acephate, azinphos-methyl, chlorpyrifos, chlorfenvinphos, diazinon, dichlorvos, dimethylvinphos, dioxabenzofos, dicrotophos, dimethoate, disulfoton, ethion, EPN, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, primiphos-ethyl, pyraclofos, pyridaphenthion, sulprophos, triazophos, trichlorfon; tetrachlorvinphos, vamidothion
carbamates such as alanycarb, benfuracarb, bendiocarb, carbaryl, carbofuran, carbosulfan, fenoxycarb, furathiocarb, indoxacarb, methiocarb, methomyl, oxamyl, pirimicarb, propoxur, thiodicarb, triazamate;
pyrethroids such as bifenthrin, cyfluthrin, cycloprothrin, cypermethrin, deltamethrin, esfenvalerate, ethofenprox, fenpropathrin, fenvalerate, cyhalothrin, lambda-cyhalothrin, permethrin, silafluofen, tau-fluvalinate, tefluthrin, tralomethrin, alpha-cypermethrin, zeta-cypermethrin, permethrin;
arthropodal growth regulators: a) chitin synthesis inhibitors, e.g. benzoylureas such as chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists such as halofenozide, methoxyfenozide, tebufenozide; c) juvenoids such as pyriproxyfen, methoprene, fenoxycarb; d) lipid biosynthesis inhibitors such as spirodiclofen;
neonicotinoids such as flonicamid, clothianidin, dinotefuran, imidacloprid, thiamethoxam, nitenpyram, nithiazin, acetamiprid, thiacloprid;
further unclassified insecticides such as abamectin, acequinocyl, acetamiprid, amitraz, azadirachtin, bensultap bifenazate, cartap, chlorfenapyr, chlordimeform, cyromazine, diafenthiuron, dinetofuran, diofenolan, emamectin, endosulfan, ethiprole, fenazaquin, fipronil, formetanate, formetanate hydrochloride, gamma-HCH hydramethylnon, imidacloprid, indoxacarb, isoprocarb, metolcarb, pyridaben, pymetrozine, spinosad, tebufenpyrad, thiamethoxam, thiocyclam, XMC and xylylcarb and
N-phenylsemicarbazones, as are described in EP-A 462 456 by the general formula I, in particular compounds of the general formula IV

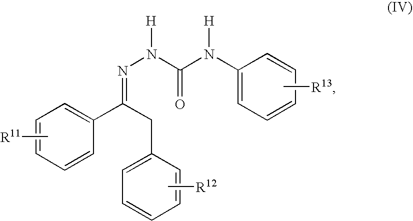

in which R11 and R12, independently of one another, are hydrogen, halogen, CN, C1-C4-alkyl, C1-C4-alkoxy, C1-C4-haloalkyl or C1-C4-haloalkoxy, and R13 is C1-C4-alkoxy, C1-C4-haloalkyl or C1-C4-haloalkoxy, e.g. compound IV in which R1 is 3-CF3 and R2 is 4-CN and R3 is 4-OCF3.

Growth regulators which can be used are e.g. chlormequat chloride, mepiquat chloride, prohexadione-calcium or those from the group of gibberellins. These include, for example, the gibberellins GA1, GA3, GA4, GA5 and GA7 etc. and the corresponding exo-16,17-dihydrogibberellins, and also the derivatives thereof, e.g. the esters with C1-C4-carboxylic acids. According to the invention, preference is given to exo-16,17-dihydro-GA5 13-acetate.

A preferred embodiment of the invention relates to the use according to the invention of hydrophobin for the preparation of aqueous active ingredient compositions of fungicides, in particular strobilurins, azoles and 6-aryltriazolo[1,5a]pyrimidines, as are described e.g. in WO 98/46608, WO 99/41255 or WO 03/004465 in each case by the general formula I (page 1, line 8 to page 11, line 45, and also compounds depicted in formula IA in conjunction with tables 1 to 44 and table A in WO 03/00465), in particular for active ingredients of the general formula V,

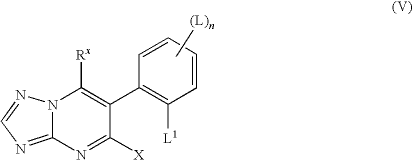

in which:
Rx is a group NR14R15, or linear or branched C1-C8-alkyl, which is optionally substituted by halogen, OH, C1-C4-alkoxy, phenyl or C3-C6-cycloalkyl, C2-C6-alkenyl, C3-C6-cycloalkyl, C3-C6-cycloalkenyl, phenyl or naphthyl, where the 4 last-mentioned radicals can have 1, 2, 3 or 4 substituents selected from halogen, OH, C1-C4-alkyl, C1-C4-haloalkoxy, C1-C4-alkoxy and C1-C4-haloalkyl;
R14, R15 independently of one another are hydrogen, C1-C8-alkyl, C1-C8-haloalkyl, C3-C10-cycloalkyl, C3-C6-halocycloalkyl, C2-C8-alkenyl, C4-C10-alkadienyl, C2-C8-haloalkenyl, C3-C6-cycloalkenyl, C2-C8-halocycloalkenyl, C2-C8-alkynyl, C2-C8-haloalkynyl or C3-C6-cycloalkynyl,
R14 and R15 together with the nitrogen atom to which they are bonded, are five- to eight-membered heterocyclyl, which is bonded via N and can comprise one, two or three further heteroatoms from the group O, N and S as ring member and/or can carry one or more substituents from the group consisting of halogen, C1-C6-alkyl, C1-C6-haloalkyl, C2-C6-alkenyl, C2-C6-haloalkenyl, C1-C6-alkoxy, C1-C6-haloalkoxy, C3-C6-alkenyloxy, C3-C6-haloalkenyloxy, (exo)-C1-C6-alkylene and oxy-C1-C3-alkyleneoxy;
L is selected from halogen, cyano, C1-C6-alkyl, C1-C4-haloalkyl, C1-C6-alkoxy, C1-C4-haloalkoxy and C1-C6-alkoxycarbonyl;
L1 is halogen, C1-C6-alkyl or C1-C6-haloalkyl and in particular fluorine or chlorine;
X is halogen, C1-C4-alkyl, cyano, C1-C4-alkoxy or C1-C4-haloalkyl and is preferably halogen or methyl and in particular chlorine.

Examples of compounds of the formula V are
5-chloro-7-(4-methylpiperidin-1-yl)-6-(2,4,6-trifluorophenyl)[1,2,4]triazolo[1,5-a]pyrimidine,
5-chloro-7-(4-methylpiperazin-1-yl)-6-(2,4,6-trifluorophenyl)[1,2,4]triazolo[1,5-a]pyrimidine,
5-chloro-7-(morpholin-1-yl)-6-(2,4,6-trifluorophenyl)[1,2,4]triazolo[1,5-a]pyrimidine,
5-chloro-7-(piperidin-1-yl)-6-(2,4,6-trifluorophenyl)[1,2,4]triazolo[1,5-a]pyrimidine,
5-chloro-7-(morpholin-1-yl)-6-(2,4,6-trifluorophenyl)[1,2,4]triazolo[1,5-a]pyrimidine,
5-chloro-7-(isopropylamino)-6-(2,4,6-trifluorophenyl)[1,2,4]triazolo[1,5-a]pyrimidine,
5-chloro-7-(cyclopentylamino)-6-(2,4,6-trifluorophenyl)[1,2,4]triazolo[1,5-a]Pyrimidine,
5-chloro-7-(2,2,2-trifluoroethylamino)-6-(2,4,6-trifluorophenyl)[1,2,4]triazolo[1,5-a]pyrimidine,
5-chloro-7-(1,1,1-trifluoropropan-2-ylamino)-6-(2,4,6-trifluorophenyl)[1,2,4]triazolo-[1,5-a]pyrimidine,
5-chloro-7-(3,3-dimethylbutan-2-ylamino)-6-(2,4,6-trifluorophenyl)[1,2,4]triazolo-[1,5-a]pyrimidine,
5-chloro-7-(cyclohexylmethyl)-6-(2,4,6-trifluorophenyl)[1,2,4]triazolo[1,5-a]pyrimidine,
5-chloro-7-(cyclohexyl)-6-(2,4,6-trifluorophenyl)[1,2,4]triazolo[1,5-a]pyrimidine,
5-chloro-7-(2-methylbutan-3-yl)-6-(2,4,6-trifluorophenyl)[1,2,4]triazolo[1,5-a]pyrimidine,
5-chloro-7-(3-methylpropan-1-yl)-6-(2,4,6-trifluorophenyl)[1,2,4]triazolo[1,5-a]pyrimidine,
5-chloro-7-(4-methylcyclohexan-1-yl)-6-(2,4,6-trifluorophenyl)[1,2,4]triazolo[1,5-a]Pyrimidine,
5-chloro-7-(hexan-3-yl)-6-(2,4,6-trifluorophenyl)[1,2,4]triazolo[1,5-a]pyrimidine,
5-chloro-7-(2-methylbutan-1-yl)-6-(2,4,6-trifluorophenyl)[1,2,4]triazolo[1,5-a]pyrimidine,
5-chloro-7-(3-methylbutan-1-yl)-6-(2,4,6-trifluorophenyl)[1,2,4]triazolo[1,5-a]pyrimidine,
5-chloro-7-(1-methylpropan-1-yl-6-(2,4,6-trifluorophenyl)[1,2,4]triazolo[1,5-a]Pyrimidine,
5-methyl-7-(4-methylpiperidin-1-yl)-6-(2,4,6-trifluorophenyl)[1,2,4]triazolo[1,5-a]pyrimidine,
5-methyl-7-(4-methylpiperazin-1-yl)-6-(2,4,6-trifluorophenyl)[1,2,4]triazolo[1,5-a]pyrimidine,
5-methyl-7-(morpholin-1-yl)-6-(2,4,6-trifluorophenyl)[1,2,4]triazolo[1,5-a]pyrimidine,
5-methyl-7-(piperidin-1-yl)-6-(2,4,6-trifluorophenyl)[1,2,4]triazolo[1,5-a]pyrimidine,
5-methyl-7-(morpholin-1-yl)-6-(2,4,6-trifluorophenyl)[1,2,4]triazolo[1,5-a]pyrimidine,
5-methyl-7-(isopropylamino)-6-(2,4,6-trifluorophenyl)[1,2,4]triazolo[1,5-a]pyrimidine,
5-methyl-7-(cyclopentylamino)-6-(2,4,6-trifluorophenyl)[1,2,4]triazolo[1,5-a]pyrimidine,
5-methyl-7-(2,2,2-trifluoroethylamino)-6-(2,4,6-trifluorophenyl)[1,2,4]triazolo[1,5-a]Pyrimidine,
5-methyl-7-(1,1,1-trifluoropropan-2-ylamino)-6-(2,4,6-trifluorophenyl)[1,2,4]triazolo-[1,5-a]pyrimidine,
5-methyl-7-(3,3-dimethylbutan-2-ylamino)-6-(2,4,6-trifluorophenyl)[1,2,4]triazolo-[1,5-a]pyrimidine,
5-methyl-7-(cyclohexylmethyl)-6-(2,4,6-trifluorophenyl)[1,2,4]triazolo[1,5-a]pyrimidine,
5-methyl-7-(cyclohexyl)-6-(2,4,6-trifluorophenyl)[1,2,4]triazolo[1,5-a]pyrimidine,
5-methyl-7-(2-methylbutan-3-yl)-6-(2,4,6-trifluorophenyl)[1,2,4]triazolo[1,5-a]pyrimidine, 5-methyl-7-(3-methylpropan-1-yl)-6-(2,4,6-trifluorophenyl)[1,2,4]triazolo[1,5-a]pyrimidine,
5-methyl-7-(4-methylcyclohexan-1-yl)-6-(2,4,6-trifluorophenyl)[1,2,4]triazolo[1,5-a]pyrimidine,
5-methyl-7-(hexan-3-yl)-6-(2,4,6-trifluorophenyl)[1,2,4]triazolo[1,5-a]pyrimidine,
5-methyl-7-(2-methylbutan-1-yl)-6-(2,4,6-trifluorophenyl)[1,2,4]triazolo[1,5-a]pyrimidine, 5-methyl-7-(3-methylbutan-1-yl)-6-(2,4,6-trifluorophenyl)[1,2,4]triazolo[1,5-a]pyrimidine and 5-methyl-7-(1-methylpropan-1-yl)-6-(2,4,6-trifluorophenyl)[1,2,4]triazolo[1,5-a]pyrimidine.

A further preferred embodiment of the invention relates to the use of hydrophobin as penetration intensifier for producing aqueous active ingredient compositions of insecticides, in particular of arylpyrroles such as chlorfenapyr, of pyrethroids such as bifenthrin, cyfluthrin, cycloprothrin, cypermethrin, deltamethrin, esfenvalerate, ethofenprox, fenpropathrin, fenvalerate, cyhalothrin, lambda-cyhalothrin, permethrin, silafluofen, tau-fluvalinate, tefluthrin, tralomethrin, alpha-cypermethrin, zeta-cypermethrin and permethrin, of neonicotinoids and of semicarbazones of the formula IV, of fipronil.

In one embodiment of the present invention, the use of hydrophobin as penetration intensifiers leads to a reduction in the concentration of active ingredients required for the desired effect to be achieved by 1%, 2%, 3%, 4%, 5%, %, 7%, 8%, 9%, 10%, preferably 11%, 12%, 13%, 14%, 15%, 16%, 18%, 20%, particularly preferably 22%, 25%, 30%, 35%, 40%, 45%, 50%, in particular 60%, 70%, 80%, 90%.

In one embodiment of the present invention, firstly a phosphate-buffered solution is applied to the surface, or phase boundary, to be treated. Hydrophobin in a concentration of from 0.01 to 0.2 percent by weight is dissolved in 50 mM NaH2PO4 with pH 7.5.

Following incubation with the hydrophobin solution, the application of the preparation comprising at least one active ingredient takes place.

The present invention further provides a method for the improved absorption of active ingredients upon topical application, wherein hydrophobin is applied
a) before the active ingredients
   or
b) at the same time as the active ingredients.

The present invention further provides a method for producing a composition for the improved absorption of active ingredients upon topical application, wherein hydrophobin in solid form, in solution or in dispersion in an organic or in an inorganic medium is introduced into a preparation comprising at least one active ingredient.

### EXAMPLES

### Example 1a

The background is the consideration that incubation of the cells with the antioxidatively effective reference substances, under the influence of hydrophobin A (SEQ ID NO: 20 from WO2007/14897 and herein) or B (SEQ ID NO: 26 from WO2007/14897; SEQ ID NO: 36 herein).

as penetration intensifier leads to an increased antioxidative potential. Besides the cultures which have been cultivated with reference substances and without penetration intensifiers, the controls used were untreated cultures and vehicle-treated cultures.

To find the dose, the concentrations which can be used were tested prior to the start of the main experiment by means of a cytotoxicity assay (here by MTT conversion).

On account of these preliminary experiments, a concentration of 0.001% was selected for the determination of the influence of the proteins on the antioxidative capacity of vitamin C, tocopheryl acetate and quercetin and also with regard to an economic use.

The reference substances used were vitamin E (alpha-tocopheryl acetate), vitamin C (Mg ascorbyl phosphate) and quercetin. The test cells used were normal dermal connective tissue cells (fibroblasts) since these produced good signal strengths in the evaluation method.

To ascertain the antioxidative properties of the solutions, the NHDF (normal human dermal fibroblast) cultures were sown out on 48-well culture vessels and cultivated until the culture surface was completely covered. The investigations were then carried out with these random cultures. Firstly, the cultures were treated for 24 h with the test solutions. Then, the medium (incl. test solutions) was removed, the cultures were washed with buffer and incubated with the fluorescent dye (DCFH). Then, to remove any unabsorbed dye, the samples were washed several times and the cells were treated with the colorless assay medium. The plates containing the cells were inserted into a fluorescence reader and the measurement was started with an introductory phase without stress. As a result of adding H₂O₂, intracellular, free radicals were then repeatedly induced which react with the dye to give a fluorescent derivative. However, if the free radicals are quenched beforehand by antioxidants, the formation of fluorescent derivatives is prevented or reduced.

Low fluorescence thus indicates high antioxidative capacity. These experiments were carried out with in each case 6 replicates in three independent runs.

The results of the investigations are then depicted as graphics.

Over the entire run time of 135 min, for the purposes of clarity, an instant photograph was taken after every 90 min (the complete images are shown in the annex). The y axis here represents the oxidative stress in the cells as the fluorescence which is emitted when free radicals react with the intracellular dye DCFH. This means that a low bar symbolizes low oxidative stress and thus high antioxidative capacity of the cells as a result of supplementation. All fluorescence values were corrected with the protein contents of the cultures following conclusion of the measurements (ascertained with Coomassie stain). This compensates for fluctuations in the cell number, which would also cause fluctuations in the fluorescence (on account of the varying amount of dye). Data that have been adjusted for cell count are thus shown.

Tocopheryl acetate on its own has no antioxidative potential with the cell line used in the investigations carried out.

Nevertheless, in two of three runs, reduced oxidative stress is observed in the case of the combinations hydrophobin A/tocopheryl acetate and hydrophobin B/tocopheryl acetate. This can be interpreted as a weak positive effect of the proteins.

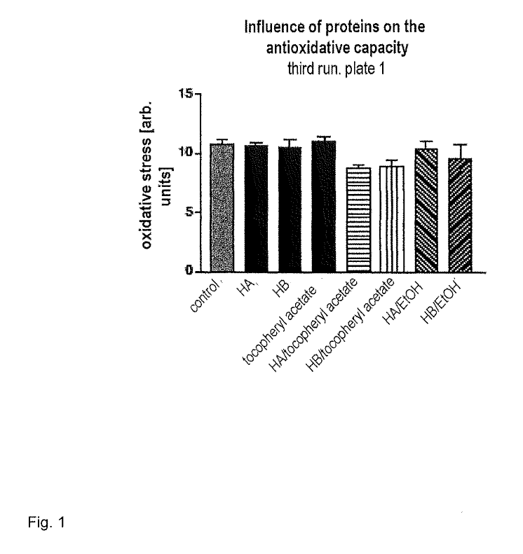

In two of three runs, the hydrophobins on their own bring about slightly reduced oxidative stress which can only in part be responsible for the reduction in oxidative stress in the combinations containing tocopheryl acetate. In the third run, this effect is the most clear. The proteins on their own exhibit no reduction in oxidative stress in this run whereas the combinations of the proteins and tocopherol acetate exhibit a drop (FIG. 1).

### Example 1b

As a further substance with antioxidative potential, quercetin was tested, in its effect on the reduction of oxidative stress under experimental conditions, materials and methods as in example 1a).

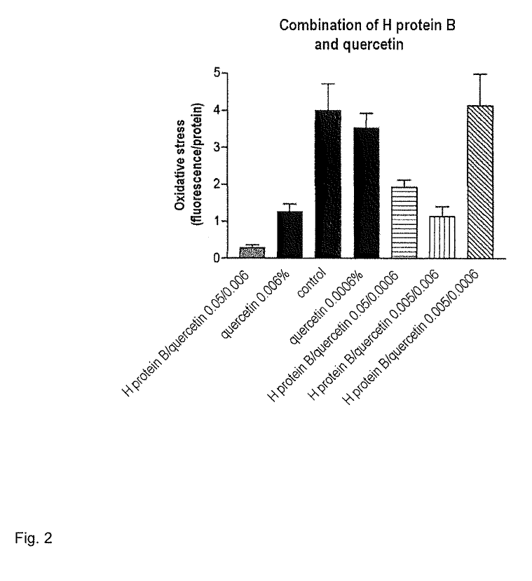

The results (FIG. 2) show that the effect of quercetin in combination with hydrophobin protein B was improved (H protein B 0.05%, combined with quercetin 0.0006%).

### Example 2

### Improved Absorption and Binding of the Dyes in Hair Tints by Hydrophobin Treated Hair

In this test, European natural hair blond from Kerling International Haarfabrik GmbH was used.

In the case of the hair tints, standard commercial preparations (stage 1) were tested.

### Test Variation 1

Preparation of hydrophobin A or B with a concentration of 0.01 to 0.2 percent by weight. Solvent is 50 mM NaH2PO₄ with pH 7.5. In order to increase the rate of the dissolution, it is dissolved at room temperature for 1 h using a magnetic stirrer.

Additionally, a comparison suspension without hydrophobin is prepared.

Incubation of the hair for 1 hour at 32° C. with subsequent rinsing with drinking water and drying.

Application of the hair tint in accordance with manufacturer's instructions.

Repeated washing (1 minute lathering, 1 minute rinsing, drying at room temperature) with a Penaten baby shampoo solution (1%), and subsequent assessment in the dried state.

### Test Variation 2

Preparation of hydrophobin A or B with a concentration of 0.01 to 0.2 percent by weight.

Solvent is 50 mM NaH2PO₄ with pH 7.5. In order to increase the rate of the dissolution, this is dissolved at room temperature for 1 h using a magnetic stirrer.

Additionally, a comparison suspension without hydrophobin is prepared.

Incubation of the hair for 1 hour at 32° C. with subsequent direct drying at 50° C. using a hair dryer.

Rinse hair with drinking water and dry at room temperature.

Application of the hair tint in accordance with the manufacturer's instructions.

Repeated washing (1 minute lathering, 1 minute rinsing, drying at room temperature) with a Penaten baby shampoo solution (1%), and subsequent assessment in the dried state.

Particularly in the case of the first test variation, it was possible to observe increased binding and dye absorption in the case of hair treated with hydrophobin A and B.

### Example 3

### Improved Skin Penetration of Lactic Acid as a Result of Treatment with Hydrophobin

### Preparation of the Hydrophobin Solutions:

1. Preparation of a 1% strength solution of hydrophobin A and B in Millipore water Dissolution of the hydrophobin by vigorous stirring on a magnetic stirrer at 900 rpm for 45 minutes.
2. Preparation of 1 ml of a ready-to-use hydrophobin solution:
   800 μl Millipore water
      +100 μl 10-fold buffer (500 mM Tris, 10 mM CaCl2, pH 8)
      +100 μl hydrophobin solution

This corresponds to a 0.1% strength hydrophobin solution in 50 mM Tris 1 mM CaCl2 and pH 8

### Experimental Procedure:

1. Frozen and dehaired pigskin (not blanched) was defrosted at room temperature.
   Marking of the application fields measuring ca. 2 cm×2 cm
   Application of the following solutions (stripping):
   control (without solution)

### 100 μl 0.1% H*A

### 100 μl 0.1% H*B

2. Incubation of the solutions for 1.5 hours at RT

In order to remove any buffer salts present, all of the application fields were rinsed with 2×500 μl of Millipore water and dabbed with a paper towel.
3. Application of in each case 100 μl of 15% strength lactic acid to all application fields

Incubation of the solutions for 10 minutes at room temperature

Removal of the excess lactic acid using a paper towel

Measurement of the pH of the pigskin on the respective application fields. Triple measurements were carried out and the mean was used. The water present after a pH measurement was dabbed off using a paper towel. A skin pH meter PH 905 from Courage & Khazaka was used.

Removal of the uppermost loose cell layer using a Corneofix adhesive strip from Courage & Khazaka

Renewed measurement of the pH

Overall, a Corneofix adhesive strip was used 3 times and the pH measured thereafter.

| pH | without | 1x stripping | 2x stripping | 3x stripping |
|---|---|---|---|---|
| Control | 2.83 | 3.52 | 3.62 | 3.67 |
| H*A | 2.62 | 3.08 | 3.39 | 3.63 |
| H*B | 2.64 | 2.89 | 3.08 | 3.26 |

### Result:

It can be seen that a coating with hydrophobin, particularly with hydrophobin B (H*B), but also with hydrophobin A (H*A) and subsequent lactic acid treatment led to a lower pH of the skin in the lower skin layers (after stripping) than did the control without hydrophobin.

This result clearly shows that hydrophobin leads to improved penetration of lactic acid into the skin, hydrophobin thus serves as a penetration enhancer for this substance.

All references cited above are incorporated by reference herein in their entirety for all useful purposes.

1-14. (canceled)

15. A method for intensifying penetration of a substance through a phase boundary comprising utilizing a hydrophobin to intensify the penetration of the substance through the phase boundary.

16. The method of claim 15, wherein the penetration of active ingredients is promoted.

17. The method of claim 15, wherein the penetration of effector molecules is promoted.

18. The method of claim 15, wherein the penetration of the substance further is intensified by utilizing the hydrophobin and at least one additional penetration intensifier.

19. The method of claim 18, wherein the at least one additional penetration intensifier is selected from the group consisting of DMSO, SDS (sodium dodecylsulfate), dimethylformamide, N-methylformamide, mono- or polyhydric alcohols, saturated and unsaturated fatty alcohols having 8 to 10 carbon atoms, hydrocarbons, alkanes, esters, azones, propylene glycol, chitosan, saturated and unsaturated fatty acids, fatty acid esters having up to 24 carbon atoms or dicarboxylic acid diesters having up to 24 carbon atoms, phosphate derivatives, terpenes, urea and its derivatives and ethers, bile salts, polyoxyethylenes, EDTA, nerolidol, limonene oxides and phospholipids.

20. A method for producing a composition for the improved absorption of active ingredients upon topical application, the method comprising incorporating hydrophobin into the composition.

21. The method of claim 28, wherein the composition is a crop protection composition.

22. The method of claim 28, wherein the composition is a semisolid medicament form.

23. The method of claim 30, wherein the semisolid medicament form is selected from the group consisting of an ointment, a cream, a gel and a paste.

24. The method of claim 28, wherein the composition is a membrane, a matrix or a plaster.

25. The method of claim 28, wherein the composition is a dermatological preparation.

26. The method of claim 28, wherein the composition is a cosmetic preparation.

27. The method of claim 28, wherein the composition is a hair cosmetic, skin cosmetic or dental cosmetic preparation.

28. A composition comprising an active ingredient and a hydrophobin, wherein the hydrophobin intensifies penetration of the active ingredient.

29. The composition of claim 37, wherein the hydrophobin intensifies the penetration of the active ingredient through a phase boundary.

30. The composition of claim 37, wherein the hydrophobin intensifies the penetration of effector molecules.

31. The composition of claim 37, in addition to the active ingredient and the hydrophobin, further comprises at least one additional penetration intensifier.

32. The composition of claim 37, which is a crop protection composition.

33. The composition of claim 37, which is a dermatological preparation.

34. The composition of claim 37, which is a cosmetic preparation.


SEQUENCE LISTING (36 sequences)

SEQ ID NO: 1 - DNA, 405 bp, Aspergillus nidulans
  CDS (1)..(405): basf-dewA hydrophobin
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 2 - protein, 135 aa, Aspergillus nidulans
  basf-dewA hydrophobin
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 3 - DNA, 471 bp, Aspergillus nidulans
  CDS (1)..(471): basf-rodA hydrophobin
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 4 - protein, 157 aa, Aspergillus nidulans
  basf-rodA hydrophobin
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 5 - DNA, 336 bp, Artificial Sequence
  CDS (1)..(336): basf-hypA from chemically synthesized polynucleotide
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 6 - protein, 112 aa, Artificial Sequence
  basf-hypA from chemically synthesized polynucleotide
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 7 - DNA, 357 bp, Artificial Sequence
  CDS (1)..(357): basf-hypB from chemically synthesized polynucleotide
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 8 - protein, 119 aa, Artificial Sequence
  basf-hypB from chemically synthesized polynucleotide
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 9 - DNA, 408 bp, Schyzophyllum commune
  CDS (1)..(408): basf-sc3 hydrophobin, cDNA template
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 10 - protein, 136 aa, Schyzophyllum commune
  basf-sc3 hydrophobin, cDNA template
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 11 - DNA, 483 bp, Artificial Sequence
  CDS (1)..(483): basf-BASF1 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 12 - protein, 161 aa, Artificial Sequence
  basf-BASF1 from chemically synthesized polynucleotide
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 13 - DNA, 465 bp, Artificial Sequence
  CDS (1)..(465): basf-BASF2 from chemically synthesized polynucleotide
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 14 - protein, 155 aa, Artificial Sequence
  basf-BASF2 from chemically synthesized polynucleotide
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 15 - DNA, 882 bp, Bacillus subtilis
  CDS (1)..(882): basf-yaad yaaD
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 16 - protein, 294 aa, Bacillus subtilis
  basf-yaad yaaD
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 17 - DNA, 591 bp, Bacillus subtilis
  CDS (1)..(591): basf-yaae yaaE with Gly insert at position 2
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta

SEQ ID NO: 18 - protein, 197 aa, Bacillus subtilis
  basf-yaae yaaE with Gly insert at position 2
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 19 - DNA, 1329 bp, Artificial Sequence
  CDS (1)..(1329): basf-yaad-Xa-dewA-his fusion of Bacillus subtilis yaaD and N-terminal factor Xa proteinase cleavage site and Aspergillus nidulans hydrophobin dewA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 20 - protein, 443 aa, Artificial Sequence
  basf-yaad-Xa-dewA-his fusion of Bacillus subtilis yaaD and N-terminal factor Xa proteinase cleavage site and Aspergillus nidulans hydrophobin dewA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 21 - DNA, 1395 bp, Artificial Sequence
  CDS (1)..(1395): basf-yaad-Xa-rodA-his fusion of Bacillus subtilis yaaD and N-terminal factor Xa proteinase cleavage site and Aspergillus nidulans hydrophobin rodA and his6
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 22 - protein, 465 aa, Artificial Sequence
  basf-yaad-Xa-rodA-his fusion of Bacillus subtilis yaaD and N-terminal factor Xa proteinase cleavage site and Aspergillus nidulans hydrophobin rodA and his6
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 23 - DNA, 1407 bp, Artificial Sequence
  CDS (1)..(1407): basf-yaad-Xa-BASF1-his fusion of Bacillus subtilis yaaD and N-terminal factor Xa proteinase cleavage site and artificial hydrophobin BASF1; BASF1 from chemically synthesized polynucleotide
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 24 - protein, 469 aa, Artificial Sequence
  basf-yaad-Xa-BASF1-his fusion of Bacillus subtilis yaaD and N-terminal factor Xa proteinase cleavage site and artificial hydrophobin BASF1; BASF1 from chemically synthesized polynucleotide
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 25 - DNA, 28 bp, Artificial Sequence
  Chemically synthesized Hal570 primer
gcgcgcccatggctcaaacaggtactga

SEQ ID NO: 26 - DNA, 28 bp, Artificial Sequence
  Chemically synthesized Hal571 primer
gcagatctccagccgcgttcttgcatac

SEQ ID NO: 27 - DNA, 30 bp, Artificial Sequence
  Chemically synthesized Hal572 primer
ggccatgggattaacaataggtgtactagg

SEQ ID NO: 28 - DNA, 33 bp, Artificial Sequence
  Chemically synthesized Hal573 primer
gcagatcttacaagtgccttttgcttatattcc

SEQ ID NO: 29 - DNA, 38 bp, Artificial Sequence
  Chemically synthesized KaM416 primer
gcagcccatcagggatccctcagccttggtaccagcgc

SEQ ID NO: 30 - DNA, 50 bp, Artificial Sequence
  Chemically synthesized KaM417 primer
cccgtagctagtggatccattgaaggccgcatgaagttctccgtctccgc

SEQ ID NO: 31 - DNA, 45 bp, Artificial Sequence
  Chemically synthesized KaM434 primer
gctaagcggatccattgaaggccgcatgaagttctccattgctgc

SEQ ID NO: 32 - DNA, 30 bp, Artificial Sequence
  Chemically synthesized KaM435 primer
ccaatggggatccgaggatggagccaaggg

SEQ ID NO: 33 - DNA, 38 bp, Artificial Sequence
  Chemically synthesized KaM418 primer
ctgccattcaggggatcccatatggaggagggagacag

SEQ ID NO: 34 - DNA, 32 bp, Artificial Sequence
  Chemically synthesized KaM464 primer
cgttaaggatccgaggatgttgatgggggtgc

SEQ ID NO: 35 - DNA, 35 bp, Artificial Sequence
  Chemically synthesized KaM465 primer
gctaacagatctatgttcgcccgtctccccgtcgt

SEQ ID NO: 36 - protein, 187 aa, Artificial Sequence
  fusion protein yaad40-Xa-dewA-his
MAQTGTERVKRGMAEMQKGGVIMDVINAEQAKIAEEAGAVIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH